(19) United States
(12) Patent Application Publication
(10) Pub. No.: US 2024/0161876 A1
(43) Pub. Date: May 16, 2024

(54) METHOD FOR IDENTIFYING DELETERIOUS GENETIC MUTATIONS

(71) Applicant: University of Macau, Macau (CN)

(72) Inventors: San Ming WANG, Macau (CN); Benjamin TAM, Macau (CN)

(21) Appl. No.: 18/551,601

(22) PCT Filed: Mar. 24, 2021
(86) PCT No.: PCT/CN2021/082783

§ 371 Date: Sep. 21, 2023

(51) Int. Cl. G16B 45/00; G16B 5/00

(52) U.S. Cl. CPC G16B 45/00; G16B 5/00

(57) ABSTRACT

A method for identifying deleterious genetic mutations, which method relates to the technical field of biomolecules. The method comprises: respectively converting, into density maps, obtained Ramachandran plots of wild-type proteins, benign protein variants, pathogenic protein variants, and proteins to be identified; dividing each density map into a plurality of regions, and by using the density maps of the benign protein variants, the pathogenic protein variants and the wild-type proteins as a reference, calculating an average density and a standard deviation of each region; if the deviation between the density of the proteins to be identified in each region and the average density of the region exceeds the standard deviation, marking the region as a density deviation region of the proteins to be identified; and on the basis of deviation data of the density deviation region of the proteins to be identified, determining mutations of the proteins to be identified. By means of the method, deleteriousness of unknown mutations can be identified with high throughput, thereby providing an approach for the study of gene mutations associated with cancer and other diseases, and diagnostic methods and therapeutic drugs therefor. The method has broad application prospects.

### TECHNICAL FIELD

The present disclosure relates to the technical field of biomolecules, and in particular to a method for identifying deleterious genetic mutations.

### BACKGROUND ART

Clarification of the pathogenic impact of genetic variation is very challenging, as it relies on the combinational evidence derived from clinic, biostatistics, molecules and experiments. Recent application of new DNA sequencing technologies has drastically increased the power for genetic study, resulting in the accumulation of massive genetic variation data at population level. The vast quantity of accumulated variation data has far surpassed the capacity of the current annotation system.

The situation is well exemplified by the genetic variants collected from the cancer predisposition gene BRCA1 and BRCA2: 80% of 40000 or more genetic variants identified from the two genes remain uncharacterized; and in the characterized variants, 30% or more of the BRCA1 variants and 40% of the BRCA2 variants are classified as Variant of Uncertain Significance (VUS) due to the lack of evidence of pathogenicity.

Therefore, there is an urgent need to develop a new method to address the problem of variant analyzing.

### SUMMARY OF THE INVENTION

An object of the present disclosure is to provide a method for identifying deleterious genetic mutations.

The present disclosure is implemented in this way:

In a first aspect, the present disclosure provides a method for identifying deleterious genetic mutations, wherein the method comprises the following steps:
respectively converting, into corresponding density maps, obtained Ramachandran plot of wild-type proteins, obtained Ramachandran plot of benign protein variants, obtained Ramachandran plot of pathogenic protein variants and obtained Ramachandran plot of proteins to be identified;
respectively dividing the density map of the wild-type proteins, the density map of the benign protein variants, the density map of the pathogenic protein variants and the density map of the proteins to be identified into n or more regions in the same dividing manner; by using N cases of the density map of the benign protein variants, the density map of the pathogenic protein variants and the density map of the wild-type proteins as a reference, calculating an average density and a standard deviation of each of the regions, wherein n≥2 and N≥2;
comparing the density of the proteins to be identified in each of the regions with the corresponding average density of the region, if the deviation between the density of the proteins to be identified in the region and the average density of the region exceeds the corresponding standard deviation of the region, marking the region as a density deviation region of the proteins to be identified;
and on the basis of the proportion or number of the density deviation regions of the proteins to be identified, determining the mutation of the proteins to be identified, and if the proportion or number of the density deviation regions of the proteins to be identified >a set threshold, then the mutation of the proteins to be identified is determined as a deleterious variation; and if the proportion or number of the density deviation regions of the proteins to be identified≤ the set threshold, then the mutation of the proteins to be identified is determined as a undefined variation.

In a second aspect, the present disclosure provides a device for identifying deleterious genetic mutations, wherein the device comprises
a conversion module for respectively converting, into corresponding density maps, obtained Ramachandran plot of wild-type proteins, obtained Ramachandran plot of benign protein variants, obtained Ramachandran plot of pathogenic protein variants and obtained Ramachandran plot of proteins to be identified;
a calculation module for respectively dividing the density map of the wild-type proteins, the density map of the benign protein variants, the density map of the pathogenic protein variants and the density map of the proteins to be identified into n or more regions in the same dividing manner; and by using N cases of the density map of the benign protein variants, the density map of the pathogenic protein variants and the density map of the wild-type proteins as a reference, calculating an average density and a standard deviation of each of the regions, wherein n≥2 and N≥2;
a marking module for comparing the density of the proteins to be identified in each of the regions with the corresponding average density of the region, if the deviation between the density of the proteins to be identified in the region and the average density of the region exceeds the corresponding standard deviation of the region, marking the region as a density deviation region of the proteins to be identified;
and a determining module for according to the proportion or number of the density deviation regions of the proteins to be identified, determining the mutation of the proteins to be identified, and if the proportion or number of the density deviation regions of the proteins to be identified>the set threshold, then the mutation of the proteins to be identified is determined as a deleterious variation; and if the proportion or number of the density deviation regions of the proteins to be identified≤the set threshold, then the mutation of the proteins to be identified is determined as a undefined variation.

In a third aspect, the present disclosure provides an electronic equipment comprising a memory and a processor, wherein when the processor runs the computer program in the memory, the method for identifying deleterious genetic mutations of the preceding embodiments is executed.

In a fourth aspect, the embodiment of the present disclosure provides a computer readable storage medium, wherein the computer readable storage medium stores a computer program, wherein when the computer program is executed by a processor, the method for identifying deleterious genetic mutations of the preceding embodiments is implemented.

The present disclosure has the following beneficial effects:

The embodiment of the present disclosure provides a method for identifying deleterious genetic mutations, which method comprises respectively converting, into density maps, obtained Ramachandran plot of wild-type proteins, obtained Ramachandran plot of benign protein variants, obtained Ramachandran plot of pathogenic protein variants and obtained Ramachandran plot of proteins to be identified, dividing the density map into a plurality of regions, and by using the density map of the benign protein variants, the density map of the pathogenic protein variants and the density map of the wild-type proteins as a reference, calculating an average density and a standard deviation of each region; comparing the density of the proteins to be identified in each of the regions with the corresponding average density of the region, if the deviation between the density of the proteins to be identified in the region and the average density of the region exceeds the standard deviation, marking the region as a density deviation region of the proteins to be identified; and on the basis of deviation data of the density deviation region of the proteins to be identified, determining mutations of the proteins to be identified. By means of the method, deleteriousness of unknown mutations can be identified with high throughput, thereby providing an approach for the study of gene mutations associated with cancer and other diseases, and diagnostic methods and therapeutic drugs therefor. The method has broad application prospects.

### BRIEF DESCRIPTION OF THE DRAWINGS

In order to more clearly illustrate the technical solutions of the examples of the present disclosure, the drawings used in the examples will be briefly introduced below. It should be understood that the following drawings only show certain examples of the present disclosure, and therefore should not be considered as limiting the scope. For a person skilled in the art, other related drawings also can be obtained according to the drawings without creative efforts.

FIG. 1 is a flowchart of a method for identifying deleterious genetic mutations in Example 1;

FIG. 2 is an example of the wild-type P53 DBD structure and the structure affected by the mutation after 40 ns simulation in Example 2;

FIG. 3 is a Ramachandran plot of P53 in Example 2; wherein a is the Ramachandran plot of wild-type P53, comprising the dihedral angles φ and Ψ of all residues; the fluctuation density is concentrated in α-helix, β-strand, γ, δ, δ′ and PII strand regions; and there is a minor fluctuation concentration in the δ′ region; b is the 2D kernel density map of wild-type P53 converted from Ramachandran plot; c is Ramachandran plots of wild-type and variant residues of pathogenic (R175H, G245D, G245S, R248Q and R273C) variants and benign (N235S) variant; and d is density map of pathogenic variants.

### DETAILED DESCRIPTION OF EMBODIMENTS

In order to make the objects, technical solutions and advantages of examples of the present disclosure clearer, the technical solutions in the examples of the present disclosure will be clearly and completely described below. If specific conditions are not specified in the examples, conventional conditions or conditions recommended by a manufacturer are followed. The reagents or instruments used therein for which manufacturers are not specified are all conventional products that are commercially available.

It should be noted that relative terms such as terms “first” and “second” are only used to distinguish one entity or operation from another entity or operation, and do not necessarily require or imply any such actual relationship or order between the entities or operations. Furthermore, the term “include”, “comprise” or any other variation thereof is intended to cover a non-exclusive inclusion, such that a process, method, article or equipment comprising a set of elements includes not only those elements, but also includes other elements not expressly listed, or also include elements inherent in such process, method, article or equipment. Without further limitations, an element defined by the phrase “comprising a . . . ” does not exclude the presence of additional identical element in the process, method, article or equipment comprising the element.

Firstly, the embodiment of the present disclosure provides a method for identifying deleterious genetic mutations (RP-MDS), wherein the method comprises the following steps:
respectively converting, into corresponding density maps, obtained Ramachandran plot of wild-type proteins, obtained Ramachandran plot of benign protein variants, obtained Ramachandran plot of pathogenic protein variants and obtained Ramachandran plot of proteins to be identified;
respectively dividing the density map of the wild-type proteins, the density map of the benign protein variants, the density map of the pathogenic protein variants and the density map of the proteins to be identified into n or more regions in the same dividing manner; and by using N cases of the density map of the benign protein variants, the density map of the pathogenic protein variants and the density map of the wild-type proteins as a reference, calculating an average density and a standard deviation of each of the regions, wherein n≥2 and N≥2;
comparing the density of the proteins to be identified in each of the regions with the corresponding average density of the region, if the deviation between the density of the proteins to be identified in the region and the average density of the region exceeds the corresponding standard deviation of the region, marking the region as a density deviation region of the proteins to be identified;
and on the basis of the proportion or number of the density deviation regions of the proteins to be identified, determining the mutation of the proteins to be identified, and if the proportion or number of the density deviation regions of the proteins to be identified>a set threshold, then the mutation of the proteins to be identified is determined as a deleterious variation; and if the proportion or number of the density deviation regions of the proteins to be identified≤the set threshold, then the mutation of the proteins to be identified is determined as a undefined variation.

The “Ramachandran plot” herein is the same as Ramachandran conformation diagram, and is a method for schematic illustration of protein structure on the basis of dihedral angle visualization. Ramachandran plot is one of the most reliable theories for protein study on the basis of protein structure, and the difference between experiment and simulation is minimal. The concept is on the basis of the rigidity of the NC peptide bond. The method comprises: according to the minimum contact distance between non-bonded atoms in the protein, determining which conformations of two adjacent peptide units specified by pairs of dihedral angles (φ and Ψ) are allowed and which are not, and taking φ as the abscissa and Ψ the ordinate to mark same in the coordinate diagram, and the coordinate diagram is referred to as Ramachandran conformation diagram.

The “mutation” herein is the same as “variation”, and refers to the change of gene structure caused by the substitution, addition and deletion of base pair in the molecular structure of DNA.

The “wild-type” herein refers to no mutation in the gene sequence. The “benign variants” herein refer to the mutations that do not cause related diseases, and specifically include two types, i.e., benign mutation and likely benign mutation.

The “pathogenic variants” herein refer to the mutations that can cause related diseases, and specifically include two types, i.e. pathogenic and likely pathogenic mutations.

“Converting Ramachandran plot into density map” can be performed by Kernel Density Estimation.

After a series of creative efforts, the inventor proposes the above-mentioned method for identifying deleterious genetic mutations, which comprises performing qualitative analysis on the deleterious mutations by detecting the changes in the secondary structure of proteins caused by the mutations. By means of the method, a variety of unknown mutations can be rapidly identified with high throughput, which is beneficial to the study of various diseases and the development of diagnostic methods and therapeutic methods therefor.

Preferably, the method further comprises obtaining the Ramachandran plot of at least one protein of the wild-type proteins, the benign protein variants, the pathogenic protein variants and the proteins to be identified on the basis of molecular dynamics simulation method before converting the Ramachandran plot into the density map.

It should be noted that the Ramachandran plot of a protein can be obtained by directly obtaining the PDB structure corresponding to the protein; however, the PDB database does not comprise the structures of most protein variants. Therefore, the inventor combines Ramachandran plot with molecular dynamics simulation (MDS). By using the MDS, the protein structure is determined and equilibrated, and after the equilibrium is reached, the protein structure is measured using Ramachandran plot.

MDS is a computation-based atomistic simulation method and can analyze physical movement interaction of atoms and molecules during a fixed time period, and the trajectories thereof are used to determine macroscopic thermodynamics properties of the molecular structure. Currently, MDS has been widely used to analyze protein structure dynamics.

MDS alone can also be used to analyze the deleteriousness of unknown mutations (on the basis of H-bond, RMSD and RMSF), however the sensitivity of MDS alone for the analysis of unknown mutations is relatively low, especially for the protein with large molecular weight (such as, the protein with 198 amino acids). The RP-MDS method provided in the present disclosure has a stronger recognition ability, can effectively identify unknown variations regardless of the molecular weight of the protein variant, and has higher identification effectiveness and wider identification applicability.

Preferably, the method for obtaining the Ramachandran plot on the basis of molecular dynamics simulation method comprises: overlapping the Ramachandran plots corresponding to the trajectories of the protein at any 2 or more time points and using same as the Ramachandran plot of the protein obtained on the basis of molecular dynamics simulation method.

Preferably, the method for obtaining the Ramachandran plot on the basis of molecular dynamics simulation method comprises: overlapping the Ramachandran plots corresponding to the trajectories of the protein obtained every 5-100 ps during the last 1-20 ns of the protein trajectory and using same as the Ramachandran plot of the protein obtained on the basis of molecular dynamics simulation method. The above “every 5-100 ps” can be 5 ps, 10 ps, 15 ps, 20 ps, 25 ps, 30 ps, 35 ps, 40 ps, 45 ps, 50 ps, 55 ps, 60 ps, 65 ps, 70 ps, 75 ps, 80 ps, 85 ps, 90 ps, 95 ps and 100 ps. The above “1-20 ns” can be 1 ns, 2 ns, 3 ns, 4 ns, 5 ns, 6 ns, 7 ns, 8 ns, 9 ns or 10 ns.

In some embodiments, the dividing manner of the density map is not specifically limited, as long as the density map is divided into multiple regions.

Preferably, the dividing manner of the density map comprises: dividing the abscissa and ordinate of the density map at intervals of d, to obtain n₁xn₁ regions, wherein d>0 and n₁≥2.
preferably, n₁≥10;
preferably, n₁≥30;
and preferably, n₁≥32.

“N” in “N cases of the density map of the benign protein variants, the density map of the pathogenic protein variants and the density map of the wild-type proteins” herein is the total number of the density map of the benign protein variants, the density map of the pathogenic protein variants and the density map of the wild-type proteins, wherein the number ratio of the density map of the benign protein variants, the density map of the pathogenic protein variants and the density map of the wild-type proteins can be randomly assigned; preferably, N≥3, and when the average density and standard deviation are calculated, it should be ensured that the N samples contain three types of density maps, that is, the density map of the benign protein variants, the density map of the pathogenic protein variants and the density map of the wild-type proteins; and more preferably, the number ratio of the three is close to 1:1:1. In some embodiments, there is no specific limitation on the sample size of N, and the larger the sample size, the more valuable the calculated results will be. Preferably, N≥30;
preferably, N≥100;
and preferably, N≥300.

The “the proportion of the density deviation regions of the proteins” herein is the proportion of the density deviation regions of the proteins in the density map.

In some embodiments, the manner for determining the set threshold of the pathogenic variant comprises: comparing the density of the pathogenic protein variants in each of the regions with the corresponding average density of the region, if the deviation between the density of the pathogenic protein variants in the region and the average density of the region exceeds the corresponding standard deviation of the region, marking the region as a density deviation region of the pathogenic protein variants;
and constructing a probability distribution on the basis of M cases of the proportions or numbers of the density deviation regions of the pathogenic protein variants, and determining the set threshold of the pathogenic variants by a goodness of fit test, wherein M≥2.

In some embodiments, there is no specific limitation on the sample size of M, and the larger the sample size, the more valuable the calculated results will be. Preferably, M≥30;
preferably, M≥100;
and preferably, M≥300.

Preferably, the probability distribution is a normal distribution or Weibull distribution, and the test method comprises at least one of Anderson-Darling and KS-test.

Preferably, the probability distribution is a logarithmic normal distribution.

In some embodiments, the gene mutation is selected from any one of germline mutation and somatic mutation. The type of the gene mutation is selected from: at least one of base substitution mutation, deletion mutation and insertion mutation;
and preferably, the type of the gene mutation is base substitution mutation.

Optionally, the target protein is P53 protein.

Further, the present disclosure further provides a device for identifying deleterious genetic mutations, wherein the device comprises:
a conversion module for respectively converting, into corresponding density maps, obtained Ramachandran plot of wild-type proteins, obtained Ramachandran plot of benign protein variants, obtained Ramachandran plot of pathogenic protein variants and obtained Ramachandran plot of proteins to be identified;
a calculation module for respectively dividing the density map of the wild-type proteins, the density map of the benign protein variants, the density map of the pathogenic protein variants and the density map of the proteins to be identified into n or more regions in the same dividing manner; and by using N cases of the density map of the benign protein variants, the density map of the pathogenic protein variants and the density map of the wild-type proteins as a reference, calculating an average density and a standard deviation of each of the regions, wherein n≥2 and N≥2;
a marking module for comparing the density of the proteins to be identified in each of the regions with the corresponding average density of the region, if the deviation between the density of the proteins to be identified in the region and the average density of the region exceeds the corresponding standard deviation of the region, marking the region as a density deviation region of the proteins to be identified;
and a determining module for according to the proportion or number of the density deviation regions of the proteins to be identified, determining the mutation of the proteins to be identified, and if the proportion or number of the density deviation regions of the proteins to be identified>a set threshold, then the mutation of the proteins to be identified is determined as a deleterious variation; and if the proportion or number of the density deviation regions of the proteins to be identified≤the set threshold, then the mutation of the proteins to be identified is determined as a undefined variation.

Preferably, the device further comprises: an obtaining module for obtaining the Ramachandran plot of at least one protein of the wild-type proteins, the benign protein variants, the pathogenic protein variants and the proteins to be identified on the basis of molecular dynamics simulation method.

Preferably, the method for obtaining the Ramachandran plot on the basis of molecular dynamics simulation method comprises: overlapping the Ramachandran plots corresponding to the trajectories of the protein at any 2 or more time points and using same as the Ramachandran plot of the protein obtained on the basis of molecular dynamics simulation method.

Preferably, the method for obtaining the Ramachandran plot on the basis of molecular dynamics simulation method comprises: overlapping the Ramachandran plots corresponding to the trajectories of the protein obtained every 5-100 ps during the last 1-20 ns of the protein trajectory and using same as the Ramachandran plot of the protein obtained on the basis of molecular dynamics simulation method.

The embodiment of the present disclosure further provides an electronic equipment comprising a memory and a processor, wherein when the processor runs the computer program in the memory, the method for identifying deleterious genetic mutations of any one of the preceding embodiments is executed.

The electronic equipment can comprise a memory, a processor, a bus and a communication interface, and the memory, the processor and the communication interface are electrically connected to each other directly or indirectly, so as to realize data transmission or interaction. For example, the elements can realize electrical connection with each other by one or more buses or signal lines. The processor can process information and/or data related to object discrimination to execute one or more functions of the present application.

The memory can be, but not limited to, Random Access Memory (RAM), Read Only Memory (ROM), Programmable Read-Only Memory (PROM), Erasable Programmable Read-Only Memory (EPROM), Electric Erasable Programmable Read-Only Memory (EEPROM), etc.

The processor can be an integrated circuit chip having a signal processing capability. The processor can be a general-purpose processor, including Central Processing Unit (CPU), Network Processor (NP), etc., and can also be Digital Signal Processing (DSP), Application Specific Integrated Circuit (ASIC), Field-Programmable Gate Array (FPGA) or other programmable logic devices, discrete gate or transistor logic devices, discrete hardware components.

Each component in the electronic equipment can be implemented by hardware, software or a combination thereof. In practical applications, the electronic equipment can be an equipment, such as a server, a cloud platform, a mobile phone, a tablet computer, a notebook computer, a ultra-mobile personal computer (UMPC), a hand-held computer, a netbook, a personal digital assistant (PDA), a wearable electronic equipment and a virtual reality equipment, and therefore the embodiment of the present application does not limit the type of electronic equipment.

In addition, the embodiment of the present disclosure further provides a computer readable storage medium, wherein the computer readable storage medium stores a computer program, wherein when the computer program is executed by a processor, the method for identifying deleterious genetic mutations of any one of the preceding embodiments is implemented.

The characteristics and performance of the present disclosure will be further described in detail below in combination with the examples.

### Example 1

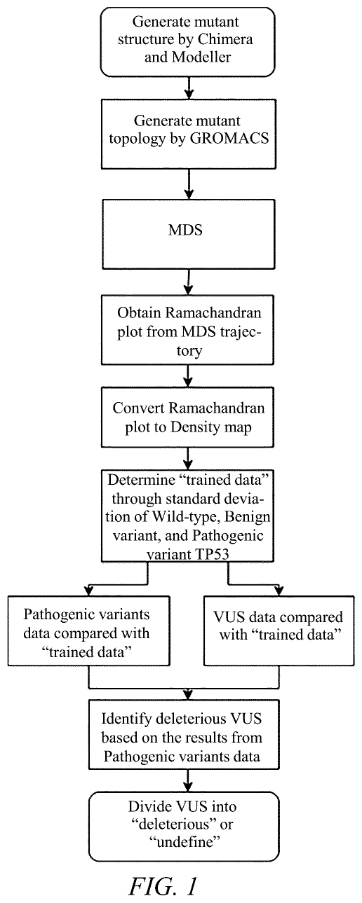

Please refer to FIG. 1. The present disclosure provides a method for identifying deleterious genetic mutations, which comprises the following steps:

### (1) Performing Molecular Dynamics Simulation:

constructing the structure of the mutant protein by using Chimera and Modeller, constructing the topology of the mutant protein by using GROMACS, and performing molecular dynamics simulation;
overlapping the Ramachandran plots corresponding to the obtained trajectories of wild-type proteins, benign protein variants, pathogenic protein and proteins to be identified every 15 ps, and using same as Ramachandran plots corresponding respectively to the wild-type proteins, the benign protein variants, the pathogenic protein and the proteins to be identified; (the benign protein variants comprise benign protein variants and likely benign protein variants);

### (2) Performing Ramachandran Plot Analysis:

respectively converting, into corresponding density maps, Ramachandran plots corresponding to the wild-type proteins, the benign protein variants, the pathogenic protein variants and the proteins to be identified obtained in step (1);
and dividing the density maps of all protein types in the same manner: dividing the abscissa and ordinate of the density map at intervals of d, to obtain 32×32 regions;
by using N cases of the density map of the benign protein variants, the density map of the pathogenic protein variants and the density map of the wild-type proteins as a reference, calculating an average density and a standard deviation of each of the regions, wherein, N≥30;
comparing the density of the pathogenic protein variants in each of the regions with the corresponding average density of the region, if the deviation between the density of the pathogenic protein variants in the region and the average density of the region exceeds the corresponding standard deviation of the region, marking the region as a density deviation region of the pathogenic protein variants; and constructing a probability distribution on the basis of M cases of the proportions of the density deviation regions of the pathogenic protein variants, and determining the set threshold of the pathogenic variants by a goodness of fit test, wherein M≥30.
comparing the density of the proteins to be identified in each of the regions with the corresponding average density of the region, if the deviation (difference) between the density of the proteins to be identified in the region and the average density of the region exceeds the corresponding standard deviation of the region, marking the region as a density deviation region of the proteins to be identified;
and on the basis of the proportion of the density deviation regions of the proteins to be identified, determining the mutation of the proteins to be identified, and if the proportion of the density deviation regions of the proteins to be identified>the set threshold, then the mutation of the proteins to be identified is determined as a deleterious variation; and if the proportion of the density deviation regions of the proteins to be identified≤the set threshold, then the mutation of the proteins to be identified is determined as a undefined variation.

### Example 2

The 42 VUS variants of P53 were determined according to the method for identifying deleterious genetic mutations provided in Example 1.

### (1) Sources of Variants and Construction of P53 Protein Variant Structures

88 TP53 variants were selected from ClinVar database, and included 38 known pathogenic variants, 8 benign/likely benign variants and 42 VUS variants. The mutations of the variants were distributed across 61 residue positions in P53 DBD region: Y107, H115, S127, A129, M133, V143, D148, P151, P152, G154, V157, Y163, Q165, T170, V173, R175, C176, R181, G187, Q192, H193, I195, R202, R213, S215, V218, Y220, G226, C229, H233, N235, C238, C242, G244, G245, M246, R248, R249, L252, I254, I255, S260, N263, L264, L265, R267, V272, R273, A276, P278, G279, D281, R282, E285, L289, K292, G293, H296, G302, S303 and N310.

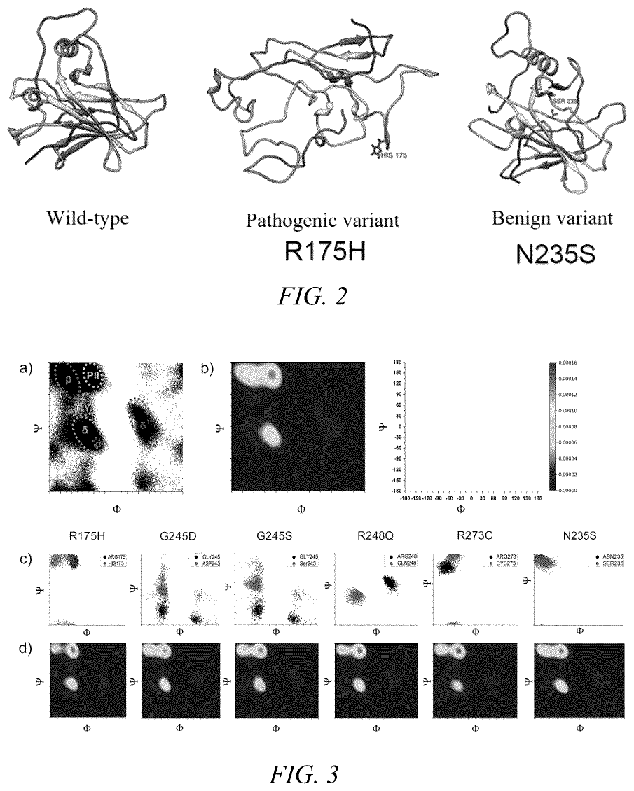

The DBD structure of P53 (PDB ID: 2OCJ, 2.05 Å, 94-313) was obtained from the PDB database. The protein structure template corresponding to each P53 variant was constructed on the basis of the DBD structure by using UCSF Chimera and Modeller software packages. Please refer to FIG. 2.

### (2) Performing Molecular Dynamics Simulation:

Each variant P53 DBD and wild-type P53 DBD structure was simulated using GROMACS molecular dynamics software. The intramolecular hydrogen bond (H-bond) and solvent surface area were simulated by the force field comparison between OPLS/AA and AMBER03. AMBER03 was selected to simulate protein complexes. Zinc ions were described by a non-bonded model that simulates 4s4p3 empty orbital. The protein structure was located in a 10×10×10 nm simulation chamber, dissolved with SPC/E water and neutralized with Cl⁻ ion. The system was optimized with steep descent algorithm before 1 ns equilibration run at 298 K and 1 bar in the NPT ensemble using Berendsen thermostat and barostat. 40 ns trajectory was simulated for the system at 298 K and 1 bar in the NPT ensemble using V-rescale thermostat and Parrinello-Rahman barostat. Verlet velocity algorithm was employed to integrate Newton's equation of motion with a time step of 2 fs. Particle Mesh Ewald method was used to treat the long-range electrostatic interactions with the cut-off distance set at 1.0 nm. LINC algorithm was used to constrain the equilibrium length of the hydrogen bond and the trajectory frame of MD was saved every 15 ps.

### (3) Ramachandran Plot Analysis:

Ramachandran plot for each variant and wild-type P53 was divided into various sub-regions: α-helices [φ, Ψ=(−63, −43)], β-strands [φ, Ψ=(−130, 140)], PII- spirals [φ, Ψ=(−45, +135)], γ′-turns [φ, Ψ=(−80, +80)], δ region [(φ, Ψ=(−63, −43)] and ε-region [φ, Ψ=(+135, +135)].

Then, the last 10 ns of the trajectory generated from MDS was used to create the Ramachandran plot of each variant and wild-type P53. Each Ramachandran plot was respectively converted to the corresponding density map by Kernel density estimation using in-house python code with a grid dimension of 32×32 (FIG. 3), and the structural deviations of each variation from the Ramachandran plot and H-bond are shown in Table 1.

TABLE 1
Structural deviations of each variation from the Ramachandran plot and H-bond

| | Native | R175H | G245D | G245S | R248Q | R273C | N235S |
|---|---|---|---|---|---|---|---|
| Global | — | 43.8 | 38.7 | 49.4 | 43.6 | 46.3 | 15.1 |
| Density | | | | | | | |
| Deviation | | | | | | | |
| (%) | | | | | | | |
| Number | 133. | 101.6 | 104.8 | 107.4 | 103.5 | 97.9 | 129.8 |
| of | | | | | | | |
| Hydrogen | | | | | | | |
| Bond | | | | | | | |

Compared to wild-type TP53, Ramachandran density map (density map converted from Ramachandran plot) show that different pathogenic variants have different structural affinities. Differences in local residue dihedral angles compared to the that of wild-type structure are shown in part c) of FIG. 3, and the Ramachandran density maps of the pathogenic protein variants are shown in part d) of FIG. 3. The mutations in P-II, α, β and δ′ regions (which are predominantly populated by folded proteins) resulted in significantly different dihedral angles from that of wild-type residues, which indicates that the mutated residue interacts with another part of the protein. The mutated residue that fluctuated at a similar dihedral angle had different interaction and therefore affected the global structure. Thus, Ramachandran density map can effectively detect the characteristic of deleterious variants.

The average density and standard deviation of each of the regions were calculated on the basis of the density maps of 8 benign protein variants, the density maps of 38 pathogenic protein variants and the density map of the wild-type P53. For each region, the density of the pathogenic variants was compared with the average density, and if the difference between the density and the average density exceeds the standard deviation, the region is marked as a density deviation region. Subsequently, the percentage of the density deviation region was calculated. A logarithmic normal distribution plot was constructed on the basis of the percentage of the density deviation region of the 38 pathogenic variants, and then the goodness of fit test was performed by A-D test and K-S test fit.

TABLE 2
Logarithmic normal distribution of 38 pathogenic variants
and goodness of fit test of logarithmic normal distribution

| | | | | | Goodness | | Decision |
|---|---|---|---|---|---|---|---|
| | M, | Scale | Lower | Upper | of fit | | at |
| | mean | sigma | 95% | 95% | tests* | P-value | level(5%) |
| Pathogenic | 3.452 | 0.241 | 3.376 | 3.526 | K-S test | 1 | Can't |
| | | | | | | | reject |
| | | | | | | | Lognormal |
| | | | | | A-D test | 0.798 | Can't |
| | | | | | | | reject |
| | | | | | | | Lognormal |

Combined with Table 2, it can be seen that pathogenic variants have a logarithmic mean of 3.452, a scale sigma of 0.241, and upper and lower boundaries at 3.376 and 3.529. Namely, variables higher than 3.376 were set as the cut-off for deleterious variant, and lower than 3.376 were defined as “undefined”.

The Ramachandran density map identified 17 of 42 (41%) VUS causing significant structural deviation (V143L, D148A, G154D, V157I Q192R, V218G, C229Y, R249S, I254V, I255N, L264P, V272M, P278R, G293R, G293W, H296Y and G302E) under the condition that it is known that the deleterious value generated by known pathogenic variants>3.376. Accordingly, we classified the 17 VUS as deleterious variants (Table 3).

TABLE 3
Detection results

| | Change* | | | RMSD | Structure deviation | |
|---|---|---|---|---|---|---|
| Genome position | Nucleotide Amino Acid | | H bond | (nm) | (%) | Impact |
| Pathogenic | | | | | | |
| Chr17:7578407 | c.523C>G | p.R175G | 125.9 | 0.251 | 29.98 | Deleterious |
| Chr17:7578403 | c.527G>A | p.C176Y | 140.3 | 0.265 | 35.35 | Deleterious |
| Chr17:7578271 | c.578A>C | p.H193P | 129.1 | 0.325 | 33.40 | Deleterious |
| Chr17:7578265 | c.584T>C | p.I195T | 135.4 | 0.257 | 35.25 | Deleterious |
| Chr17:7578211 | c.638G>A | p.R213Q | 131.0 | 0.307 | 33.89 | Deleterious |
| Chr17:7577556 | c.725G>A | p.C242Y | 135.8 | 0.337 | 30.96 | Deleterious |
| Chr17:7577551 | c.730G>A | p.G244S | 138.1 | 0.231 | 36.52 | Deleterious |
| Chr17:7577548 | c.733G>A | p.G245V | 131.9 | 0,320 | 32.03 | Deleterious |
| Chr17:7577538 | c.743G>T | p.R248L | 126.5 | 0.309 | 33.01 | Deleterious |
| Chr17:7577121 | c.817G>T | p.R273S | 141.2 | 0.285 | 31.54 | Deleterious |
| Chr17:7577121 | c.817C>G | p.R273L | 130.4 | 0.380 | 29.30 | Deleterious |
| Chr17:7577120 | c.818G>A | p.R273G | 134.3 | 0.284 | 42.87 | Deleterious |
| Chr17:7577096 | c.842A>G | p.D281G | 128.7 | 0.346 | 33.30 | Deleterious |
| Chr17:7577096 | c.842A>T | p.D281N | 133.9 | 0.295 | 33.79 | Deleterious |
| Chr17:7577084 | 0.854A>T | p.E285V | 136.4 | 0.362 | 38.48 | Deleterious |
| Chr17:7578442 | 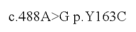 | | 90.6 | 0.639 | 38.57 | Deleterious |
| Chr17:7578406 | 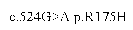 | | 101.6 | 0.720 | 43.75 | Deleterious |
| Chr17:7578190 | 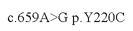 | | 84.4 | 0.649 | 43.85 | Deleterious |
| Chr17:7577550 | 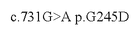 | | 104.8 | 0.580 | 38.67 | Deleterious |
| Chr17:7577548 | 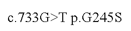 | | 107.4 | 0.650 | 49.41 | Deleterious |
| Chr17:7577538 | 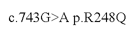 | | 103.5 | 0.582 | 43.55 | Deleterious |
| Chr17:7577120 | 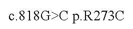 | | 97.9 | 0.685 | 46.29 | Deleterious |
| Chr17:7577093 | 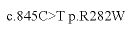 | | 87.6 | 0.775 | 44.43 | Deleterious |
| VUS | | | | | | |
| Chr17:7578503 | c.427G>T | p.V143L | 137.4 | 0.317 | 36.33 | Deleterious |
| Chr17:7578487 | c.443A>C | p.D148A | 138.9 | 0.338 | 31.84 | Deleterious |
| Chr17:7578469 | c.461G>A | p.G154D | 137.2 | 0.329 | 36.52 | Deleterious |
| Chr17:7578461 | c.469G>A | p.V1571 | 139.7 | 0.315 | 33.11 | Deleterious |
| Chr17:7578274 | c.575A>G | p.Q192R | 137.8 | 0.407 | 31.45 | Deleterious |
| Chr17:7578196 | c.653T>G | p.V218G | 135.3 | 0.283 | 36.13 | Deleterious |
| Chr17:7577595 | c.686G>A | p.C229Y | 136.4 | 0.322 | 31.84 | Deleterious |
| Chr17:7577521 | c.760A>G | p.I254V | 134.1 | 0.333 | 32.71 | Deleterious |
| Chr17:7577517 | c.764T>A | p.I25SN | 132.7 | 0.280 | 30.37 | Deleterious |
| Chr17:7577147 | c.791T>C | p.L264P | 138.7 | 0.279 | 31.15 | Deleterious |
| Chr17:7577124 | c.814G>A | p.V272M | 137.2 | 0.311 | 29.98 | Deleterious |
| Chr17:7577105 | c.833C>A | p.P278R | 131.3 | 0.367 | 40.53 | Deleterious |
| Chr17:7577061 | c.877G>A | p.G293R | 137.9 | 0.333 | 32.52 | Deleterious |
| Chr17:7577061 | c.877G>T | p.G293W | 131.1 | 0.362 | 29.59 | Deleterious |
| Chr17:7577052 | c.886C>T | p.H296Y | 132.2 | 0.360 | 30.37 | Deleterious |
| Chr17:7577033 | c.905G>A | p.G302E | 134.9 | 0.348 | 29.79 | Deleterious |

| | | | | | |
|---|---|---|---|---|---|
| Chr17:7577534 | 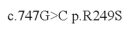 | 103.9 | 0.668 | 43.85 | Deleterious |

The test of 38 pathogenic variants showed that MDS can effectively identify deleterious variants with significant pathogenic properties (mutations marked in grayscale in Table 3), such as 8 TP53 pathogenic variants with low H-bond and high RMSD. However, for less deleterious variants, the sensitivity of MDS is insufficient. In addition to the 8 pathogenic variants identified by MDS, the method for identifying deleterious genetic mutations provided in the present disclosure was able to further detect 15 other variants which have structural deviations that can not be detected according to H-bond, RMSD, and RMSF.

Missense3D and SuSPect were used to test the known 38 pathogenic variants, 8 benign/likely benign variants and 42 VUS mentioned above. Missense 3D confirmed that 13 pathogenic variants (R175G, R175H, C176Y, H193P, R213Q, Y220C, C238R, C242Y, G245D, G245S, G245V, L265P and R273P) and 8 VUS (C176W, G187D, S215R, V218G, L252P, I255N, P278R and G279R) had underlying structural damage, and all benign variants were correctly classified. SuSPect can be used to detect all diseases associated with pathogenic variants, however, the program failed to differentiate benign and likely benign variants and classified all VUS as disease-associated variants. RP-MDS method provided in the present disclosure can effectively identify deleterious mutation, and all the benign variants were classified in the “undefined” region.

### INDUSTRIAL PRACTICABILITY

The present application discloses a method for identifying deleterious genetic mutations, which method comprises: converting, into density maps, all of obtained Ramachandran plot of wild-type proteins, obtained Ramachandran plot of benign protein variants, obtained Ramachandran plot of pathogenic protein variants and obtained Ramachandran plot of proteins to be identified; dividing the density maps into a plurality of regions, and by using the density maps of the benign protein variants, the density maps of the pathogenic protein variants and the density maps of the wild-type proteins as a reference, calculating an average density and a standard deviation of each region; comparing the density of the proteins to be identified in each of the regions with the corresponding average density of the region, if the deviation between the density of the proteins to be identified in the region and the average density of the region exceeds the standard deviation, marking the region as a density deviation region of the proteins to be identified; and on the basis of deviation data of the density deviation region of the proteins to be identified, determining mutations of the proteins to be identified.

By means of the method, deleteriousness of unknown mutations can be identified with high throughput, which provides a new approach for the study of various disease diagnostic markers and therapeutic drugs, and has broad industrial application prospects.

1. A method for identifying deleterious genetic mutations, wherein the method comprises the following steps:
respectively converting, into corresponding density maps, obtained Ramachandran plot of wild-type proteins, obtained Ramachandran plot of benign protein variants, obtained Ramachandran plot of pathogenic protein variants and obtained Ramachandran plot of proteins to be identified;
respectively dividing the density map of the wild-type proteins, the density map of the benign protein variants, the density map of the pathogenic protein variants and the density map of the proteins to be identified into n or more regions in the same dividing manner; by using N cases of the density map of the benign protein variants, the density map of the pathogenic protein variants and the density map of the wild-type proteins as a reference, calculating an average density and a standard deviation of each of the regions, wherein n≥2 and N≥2;
comparing the density of the proteins to be identified in each of the regions with the corresponding average density of the region, if the deviation between the density of the proteins to be identified in the region and the average density of the region exceeds the corresponding standard deviation of the region, marking the region as a density deviation region of the proteins to be identified;
and on the basis of the proportion or number of the density deviation regions of the proteins to be identified, determining the mutation of the proteins to be identified, and if the proportion or number of the density deviation regions of the proteins to be identified>a set threshold, then the mutation of the proteins to be identified is determined as a deleterious variation; and if the proportion or number of the density deviation regions of the proteins to be identified≤the set threshold, then the mutation of the proteins to be identified is determined as a undefined variation.

2. The method for identifying deleterious genetic mutations of claim 1, wherein the method further comprises obtaining the Ramachandran plot of at least one protein of the wild-type proteins, the benign protein variants, the pathogenic protein variants and the proteins to be identified on the basis of molecular dynamics simulation method before converting the Ramachandran plot into the density map.

3. The method for identifying deleterious genetic mutations of claim 2, wherein the method for obtaining the Ramachandran plot on the basis of molecular dynamics simulation method comprises: overlapping the Ramachandran plots corresponding to the trajectories of the protein at any 2 or more time points and using same as the Ramachandran plot of the protein obtained on the basis of molecular dynamics simulation method.

4. The method for identifying deleterious genetic mutations of claim 3, wherein the method for obtaining the Ramachandran plot on the basis of molecular dynamics simulation method comprises: overlapping the Ramachandran plots corresponding to the trajectories of the protein obtained every 5-100 ps during the last 1-20 ns of the protein trajectory and using same as the Ramachandran plot of the protein obtained on the basis of molecular dynamics simulation method.

5. The method for identifying deleterious genetic mutations of claim 1, wherein the dividing manner of the density map comprises: dividing the abscissa and ordinate of the density map at intervals of d, to obtain nixni the regions, wherein d>0 and n₁≥2;
preferably, n₁≥10;
preferably, n₁≥30;
and preferably, n₁≥32.

6. The method for identifying deleterious genetic mutations of claim 1, wherein N≥30;
preferably, N≥100;
and preferably, N≥300.

7. The method for identifying deleterious genetic mutations of claim 1, wherein comparing the density of the pathogenic protein variants in each of the regions with the corresponding average density of the region, if the deviation between the density of the pathogenic protein variants in the region and the average density of the region exceeds the corresponding standard deviation of the region, marking the region as a density deviation region of the pathogenic protein variants;
and constructing a probability distribution on the basis of M cases of the proportions or numbers of the density deviation regions of the pathogenic protein variants, and determining the set threshold of the pathogenic variants by a goodness of fit test, wherein M≥2.

8. The method for identifying deleterious genetic mutations of claim 7, wherein the probability distribution is a normal distribution or Weibull distribution.

9. The method for identifying deleterious genetic mutations of claim 8, wherein the probability distribution is a logarithmic normal distribution.

10. The method for identifying deleterious genetic mutations of claim 7, wherein the test method comprises at least one of Anderson-Darling and KS-test.

11. The method for identifying deleterious genetic mutations of one claim 7, wherein M≥30;
preferably, M≥100;
and preferably, M≥300.

12. The method for identifying deleterious genetic mutations of claim 1, wherein the gene mutation is selected from any one of germline mutation and somatic mutation.

13. The method for identifying deleterious genetic mutations of claim 12, wherein the type of the gene mutation is selected from: at least one of base substitution mutation, deletion mutation and insertion mutation;
and preferably, the type of the gene mutation is base substitution mutation.

14. The method for identifying deleterious genetic mutations of claim 1, wherein the target protein is P53 protein.

15. A device for identifying deleterious genetic mutations, wherein the device comprises:
a conversion module for respectively converting, into corresponding density maps, obtained Ramachandran plot of wild-type proteins, obtained Ramachandran plot of benign protein variants, obtained Ramachandran plot of pathogenic protein variants and obtained Ramachandran plot of proteins to be identified;
a calculation module for respectively dividing the density map of the wild-type proteins, the density map of the benign protein variants, the density map of the pathogenic protein variants and the density map of the proteins to be identified into n or more regions in the same dividing manner; by using N cases of the density map of the benign protein variants, the density map of the pathogenic protein variants and the density map of the wild-type proteins as a reference, calculating an average density and a standard deviation of each of the regions, wherein n≥2 and N≥2;
a marking module for comparing the density of the proteins to be identified in each of the regions with the corresponding average density of the region, if the deviation between the density of the proteins to be identified in the region and the average density of the region exceeds the corresponding standard deviation of the region, marking the region as a density deviation region of the proteins to be identified;
and a determining module for according to the proportion or number of the density deviation regions of the proteins to be identified, determining the mutation of the proteins to be identified, and if the proportion or number of the density deviation regions of the proteins to be identified>a set threshold, then the mutation of the proteins to be identified is determined as a deleterious variation; and if the proportion or number of the density deviation regions of the proteins to be identified≤the set threshold, then the mutation of the proteins to be identified is determined as a undefined variation.

16. The device for identifying deleterious genetic mutations of claim 15, wherein the device further comprises: an obtaining module for obtaining the Ramachandran plot of at least one protein of the wild-type proteins, the benign protein variants, the pathogenic protein variants and the proteins to be identified on the basis of molecular dynamics simulation method.

17. The device for identifying deleterious genetic mutations of claim 16, wherein the method for obtaining the Ramachandran plot on the basis of molecular dynamics simulation method comprises: overlapping the Ramachandran plots corresponding to the trajectories of the protein at any 2 or more time points and using same as the Ramachandran plot of the protein obtained on the basis of molecular dynamics simulation method.

18. The device for identifying deleterious genetic mutations of claim 17, wherein the method for obtaining the Ramachandran plot on the basis of molecular dynamics simulation method comprises: overlapping the Ramachandran plots corresponding to the trajectories of the protein obtained every 5-100 ps during the last 1-20 ns of the protein trajectory and using same as the Ramachandran plot of the protein obtained on the basis of molecular dynamics simulation method.

19. An electronic equipment, wherein the equipment comprises a memory and a processor, wherein when the processor runs a computer program in the memory, the method for identifying deleterious genetic mutations of claim 1 is executed.

20. A computer readable storage medium, wherein the computer readable storage medium stores a computer program, wherein when the computer program is executed by a processor, the method for identifying deleterious genetic mutations of claim 1 is implemented.
